(19) United States
(12) Patent Application Publication
(10) Pub. No.: US 2009/0187172 A1
(43) Pub. Date: Jul. 23, 2009

(54) METHOD FOR HARVESTING CORNEAL DONOR PLUGS FOR USE IN KERATOPHAKIA PROCEDURES

(76) Inventors: Luis Antonio Ruiz, Bogota (CO); Fritz Meisel, Kalchreuth (DE); Josef F. Bille, Heidelberg (DE)

Correspondence Address: NEIL K. NYDEGGER;NYDEGGER & ASSOCIATES, 348 Olive Street, San Diego, CA, 92103, US

(21) Appl. No.: 12/016,890

(22) Filed: Jan. 18, 2008

(51) Int. Cl. A61F 9/008

(52) U.S. Cl. 606/5

(57) ABSTRACT

A method for harvesting a plurality of corneal donor plugs for use in keratophakia procedures requires the determination of a perimeter for the plurality of plugs. Further, the method involves the selection of posterior and anterior boundaries for the plurality of donor plugs. In the method, interfaces between adjacent donor plugs are identified. Thereafter, a pulse laser beam is directed along the perimeter, the boundaries and the interfaces to establish the plurality of plugs. After the plugs are removed from the donor cornea, individual plugs are mechanically separated from one another.

### FIELD OF THE INVENTION

The present invention pertains generally to methods for performing intrastromal ophthalmic laser surgery. More particularly, the present invention pertains to the use of laser surgery to prepare corneal donor plugs. The present invention is particularly, but not exclusively, useful as a method for harvesting corneal donor plugs for use in keratophakia procedures.

### BACKGROUND OF THE INVENTION

The cornea of an eye has five (5) different identifiable layers of tissue. Proceeding in a posterior direction from the anterior surface of the cornea, these layers are: the epithelium; Bowman's membrane; the stroma; Descemet's membrane; and the endothelium. Structurally, the cornea of the eye has a thickness, between the epithelium and the endothelium that is approximately five hundred micrometers (500 μm). Within this structure, the stroma has a thickness of almost four hundred microns in the cornea.

It is well known that defective vision can be corrected by reshaping the cornea of the eye. Further, it is known that reshaping of the cornea can be accomplished in several ways. For example, the well known radial keratotomy procedure is used to establish weakened areas in the cornea which respond to internal pressure in the eye to move the cornea in its optical relationship with the retina. Another way in which vision can be corrected is by procedures which actually remove portions of the cornea to alter its optical properties. Yet another method for vision correction involves the implantation of a synthetic or natural tissue into the patient's cornea to change the curvature of the cornea. Often, implantation is the most suitable procedure for modifying refractive error for patients having thin corneas (corneas with thicknesses less than 450 micrometers). This is particularly so for patient's with thin corneas that are in need of high correction (over ten diopters). In this category of procedures, keratophakia refers to the use of a donor cornea as an implant.

In keratophakia, a plug or button of a donor's cornea is shaped to desired dimensions and is inserted into the recipient's stroma to change its curvature. Typically, a microtome is used to cut into the donor cornea. Then, a single plug is removed from the stroma of the donor cornea and is shaped for use in the keratophakia procedure. While this known method is effective, the use of the microtome typically results in an uneven cutting of the donor cornea and donor plugs with resultant irregular edges. Further, the known method obtains only a single plug from a donor cornea.

In light of the above, it is an object of the present invention to provide methods for photoablating donor corneal tissue to obtain a plurality of donor plugs from a single donor eye. Another object of the present invention is to provide methods for performing ophthalmic laser surgery that optimize the use of donor corneal tissue. Still another object of the present invention is to provide methods for harvesting corneal plugs with even surfaces. Yet another object of the present invention is to provide methods for harvesting corneal donor plugs for use in keratophakia procedures that are relatively easy to implement and comparatively cost effective.

### SUMMARY OF THE INVENTION

In accordance with the present invention, methods for harvesting a plurality of corneal donor plugs for use in keratophakia procedures are provided. Importantly, for these methods, a perimeter for the plurality of plugs is determined. Preferably, the perimeter is substantially cylindrical and is centered about an axis defined by the donor cornea. Also, a posterior boundary and an anterior boundary for the plurality of plugs are selected. Further, interfaces between adjacent plugs are identified. For the present invention, the boundaries and interfaces are substantially planar and are perpendicular to the axis. Typically, at least two, but as many as four, plugs are harvested from the stroma of a single donor cornea. Thus, with four plugs, three interfaces are identified. Preferably, each plug has a thickness of approximately 100 micrometers. As a result, each boundary is approximately 100 micrometers from the adjacent interface and each interface is approximately 100 micrometers from the adjacent interface.

In general, each method of the present invention requires the use of a laser unit that is capable of generating a so-called femtosecond laser beam. Stated differently, the duration of each pulse in the beam will be less than one picosecond. When generated, this beam is directed and focused onto a series of focal spots in the stroma of the donor cornea. The well-known result of this is a Laser Induced Optical Breakdown (LIOB) of stromal tissue at each focal spot. In particular, and as intended for the present invention, movement of the focal spot in the stroma photoablates the donor cornea along the perimeter, the boundaries, and the interfaces.

In a preferred procedure, the laser beam is operated so that each pulse of the laser beam has an energy of approximately 2.5 microJoules. This laser beam is then directed along a series of focal spots around the perimeter. Thereafter, the laser beam is modified to emit pulses having an energy of approximately 1.8 microJoules. With this lower energy level, the laser beam is directed along a series of focal spots along the posterior boundary, the interfaces, and the anterior boundary. As a result of the photoablation, the plurality of donor plugs is created in the donor cornea. After the plurality of donor plugs are created, they are removed from the donor cornea in a single cohesive unit. Each donor plug may then be mechanically separated from the others, by peeling the donor plugs apart.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a cross-sectional view of the cornea of a donor eye shown in relationship to a schematically depicted laser unit;

FIG. 2 is a perspective view of the donor cornea showing the path of LIOB to create a plurality of donor plugs in accordance with the present invention;

FIG. 3 is a cross sectional view of a recipient eye prepared with a flap for receiving a donor plug (shown in perspective view) during a keratophakia procedure;

FIG. 4 is a cross sectional view of the donor plug positioned on the recipient eye of FIG. 3 in accordance with a keratophakia procedure; and

FIG. 5 is a cross sectional view of a recipient eye after a keratophakia procedure.

### DESCRIPTION OF THE PREFERRED EMBODIMENTS

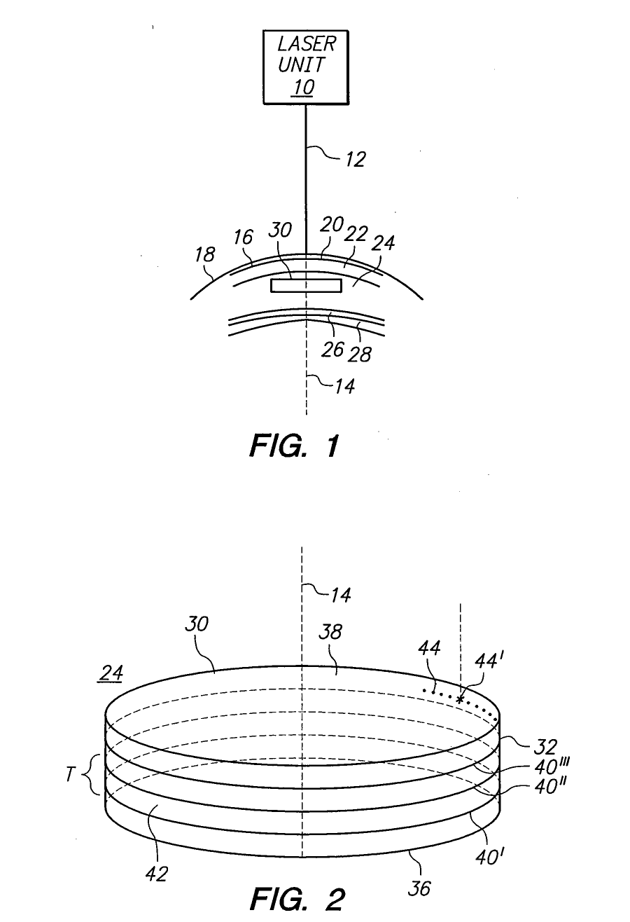

Referring initially to FIG. 1, it will be seen that the present invention includes a laser unit 10 for generating a laser beam 12. More specifically, the laser beam 12 is preferably a pulsed laser beam, and the laser unit 10 generates pulses for the beam 12 that are less than one picosecond in duration (i.e. they are femtosecond pulses). In FIG. 1, the laser beam 12 is shown being directed along the visual axis 14 and onto the cornea 16 of a donor eye 18. As shown, the cornea 16 is comprised of several distinct layers, namely, the epithelium 20, Bowman's membrane 22, the stroma 24, Descemet's membrane 26 and the endothelium 28. In the present invention, the stroma 24 is of particular importance. As shown in FIG. 1, a plurality of donor plugs 30 are identified in the stroma 24 of the donor eye 18. In the present invention, the laser beam 12 is used to photoablate the stroma 24 to remove the plurality of donor plugs 30 from the donor eye 18.

In FIG. 2, the method of photoablation is illustrated. As shown, a substantially cylindrical perimeter 32 is centered about the axis 14 to separate a plurality of donor plugs 30 from the remaining corneal tissue 16 in the stroma 24. Further, a posterior boundary 36 and an anterior boundary 38 provide the lower and upper limits of the plurality of donor plugs 30. As shown, the boundaries 36, 38 are substantially planar and are perpendicular to the axis 14. Also, three interfaces 40′, 40″, 40′″ are positioned between the boundaries 36, 38. Similar to the boundaries 36, 38, the interfaces 40′, 40″, 40′″ are substantially planar and are perpendicular to the axis 14. In FIG. 2, it can be seen that an equal distance “T” separates the posterior boundary 36 from adjacent interface 40′, the interface 40′ from adjacent interface 40″, the interface 40″ from adjacent interface 40′″, and the interface 40′″ from anterior boundary 38. Further, each adjacent boundary-interface or interface-interface pair defines a single donor plug 42. As shown, the distance “T” is the thickness for each of the four donor plugs 42, and will be determined by the requirements of the recipient eye 46. Typically, however, the thickness will be approximately 100 micrometers. Further, each of the donor plugs 42 can have an approximate diameter of as much as about 4 millimeters.

After the perimeter 32, boundaries, 36, 38 and interfaces 40′, 40″, 40′″ have been determined, photoablation is performed. Specifically, the laser unit 10 directs a pulsed laser beam 12 to a series of focal points 44 along the perimeter 32 to photoablate the corneal tissue 16 at the perimeter 32. During photoablation of the perimeter 32, each pulse of the laser beam 12 has an energy of approximately 2.5 microJoules. After photoablation of the perimeter 32 is complete, the boundaries 36, 38 and interfaces 40′, 40″, 40′″ are photoablated. During this photoablation procedure, the pulsed laser beam 12 is directed to a succession of focal points 44 along the selected boundary 36, 38 or interface 40′, 40″, 40′″. In FIG. 2, photoablation is occurring at focal point 44′. Also, the photoablation of the boundaries 36, 38 and interfaces 40′, 40″, 40′″ is performed with each pulse of the laser beam 12 having an energy of approximately 1.8 microJoules.

After photoablation is performed, the individual donor plugs 42 are created. The plurality of plugs 30 may be removed from the donor eye 18 in a single unit. Thereafter, individual plugs 42 may be separated from the plurality 30 by mechanically peeling each plug 42 away from the others. Then, each plug 42 may be used independently in a keratophakia procedure. In this manner, a plurality of plugs 30 is prepared from a single donor eye 18.

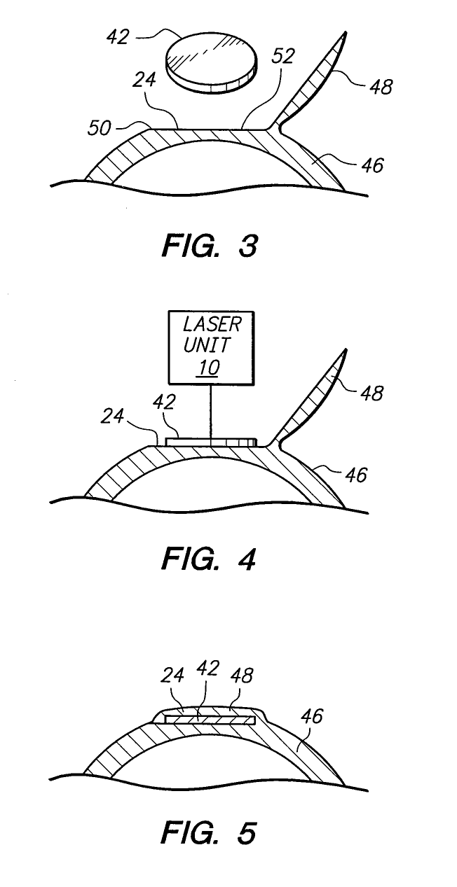

Referring to FIGS. 3-5, the use of a donor plug 42 in a keratophakia procedure is explained. As shown in FIG. 3, a recipient eye 46 is prepared by creating a flap 48 through photoablation along a substantially planar path 50. For a keratophakia procedure, the flap 48 contains the epithelium 20, Bowman's membrane 22, and a portion of the stroma 24. When the flap 48 is peeled back, the stroma 24 of the recipient eye 46 is exposed. In certain embodiments, the exposed surface 52 of the stroma 24 may be photoablated to prepare the stroma 24 for the transplant. In any event, after the stroma 24 has been prepared, the donor plug 42 is positioned on the stroma 24, as shown in FIG. 4. After the donor plug 42 is positioned on the stroma 24, the laser unit 10, preferably using an excimer laser subassembly (not shown), may be used to further photoablate the donor plug 42 and the stroma 24 of the recipient eye 46. This is done to ensure proper vision correction, and to smooth edges or correct irregularities in the donor plug 42. As shown in FIG. 5, the flap 48 is thereafter reconnected to the recipient eye 46 to encapsulate the donor plug 42 within the stroma 24. As a result, the curvature of the recipient eye 46 is changed to correct focusing deficiencies.

While the particular Method for Harvesting Corneal Donor Plugs for Use in Keratophakia Procedures as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for harvesting a plurality of corneal donor plugs for use in keratophakia procedures comprising the steps of:
providing a donor cornea;
determining a perimeter for the plurality of corneal donor plugs;
selecting a posterior boundary and an anterior boundary for the plurality of corneal donor plugs;
identifying at least one interface, with each interface separating adjacent corneal donor plugs;
generating a pulsed laser beam;
directing the pulsed laser beam along the perimeter to photoablate corneal tissue in the donor cornea;
targeting the pulsed laser beam along each boundary and each interface to photoablate corneal tissue in the donor cornea;
removing the plurality of corneal donor plugs from the donor cornea; and
peeling each corneal donor plug from the plurality of corneal donor plugs.

2. A method as recited in claim 1 wherein the perimeter is substantially cylindrical.

3. A method as recited in claim 2 wherein the boundaries and the interfaces are substantially planar and parallel.

4. A method as recited in claim 3 wherein, during the directing step, each pulse of the laser beam has an energy of approximately 2.5 microJoules to photoablate corneal tissue along the perimeter.

5. A method as recited in claim 4 wherein, during the targeting step, each pulse of the laser beam has an energy of approximately 1.8 microJoules to photoablate corneal tissue along each boundary and each interface.

6. A method as recited in claim 3 wherein the distance between each boundary and each adjacent interface is approximately 100 micrometers.

7. A method as recited in claim 6 wherein the distance between adjacent interfaces is approximately 100 micrometers, and wherein each donor plug is substantially the same size.

8. A method as recited in claim 7 wherein four corneal donor plugs are harvested from the donor cornea.

9. A method for harvesting a plurality of corneal donor plugs comprising the steps of:
providing a donor cornea defining an axis;
determining a substantially cylindrical perimeter for the plurality of corneal donor plugs, wherein said perimeter is centered about the axis;
selecting a posterior boundary and an anterior boundary for the plurality of corneal donor plugs, wherein the boundaries are substantially perpendicular to the axis;
identifying at least one interface positioned between the boundaries, wherein each interface separates adjacent corneal donor plugs, and wherein each interface is substantially perpendicular to the axis; and
generating a pulsed laser beam and directing the pulsed laser beam along the perimeter, along each boundary, and along each interface to photoablate corneal tissue in the donor cornea and to establish the plurality of donor plugs.

10. A method as recited in claim 9 further comprising the steps of:
removing the plurality of corneal donor plugs from the donor cornea; and
peeling each corneal donor plug from the plurality of corneal donor plugs.

11. A method as recited in claim 9 wherein each boundary is approximately 100 micrometers from an adjacent interface.

12. A method as recited in claim 11 wherein each interface is approximately 100 micrometers from an adjacent interface.

13. A method as recited in claim 9 wherein each pulse of the laser beam has an energy of approximately 2.5 microJoules to photoablate corneal tissue along the perimeter.

14. A method as recited in claim 13 wherein each pulse of the laser beam has an energy of approximately 1.8 microJoules to photoablate corneal tissue along each boundary and each interface.

15. A method as recited in claim 9 wherein four corneal donor plugs are harvested from the donor cornea.

16. A method for harvesting a plurality of corneal donor plugs comprising the steps of:
providing a donor cornea defining an axis;
generating a first pulsed laser beam, wherein each pulse of the first laser beam has an energy of approximately 2.5 microJoules;
directing and focusing the first beam onto a series of focal spots in the stroma of the donor corneal for Laser Induced Optical Breakdown (LIOB) of stromal tissue at each focal spot, with said series of focal spots forming a perimeter for the plurality of corneal donor plugs;
generating a second pulsed laser beam, wherein each pulse of the second laser beam has an energy of approximately 1.8 microJoules; and
directing and focusing the second beam onto a succession of focal spots in the stroma of the donor corneal for LIOB of stromal tissue at each focal spot, with said succession of focal spots forming (a) a posterior boundary and an anterior boundary for the plurality of corneal donor plugs and (b) at least one interface between adjacent donor plugs.

17. A method as recited in claim 16 further comprising the steps of:
removing the plurality of corneal donor plugs from the donor cornea; and
peeling each corneal donor plug from the adjacent corneal donor plug.

18. A method as recited in claim 16 wherein the perimeter is substantially cylindrical and is centered about the axis.

19. A method as recited in claim 18 wherein the boundaries and the interfaces are substantially planar and are perpendicular to the axis.

20. A method as recited in claim 16 wherein each donor plug has a thickness of 100 micrometers.
